(19) United States
(12) Patent Application Publication
(10) Pub. No.: US 2015/0059754 A1
(43) Pub. Date: Mar. 5, 2015

(54) SYSTEM AND METHOD FOR POWER OF BREATHING REAL-TIME ASSESSMENT AND CLOSED-LOOP CONTROLLER

(71) Applicant: KONINKLIJKE PHILIPS N.V., EINDHOVEN (NL)

(72) Inventors: Nicolas Wadih Chbat, White Plains, NY (US); Adam Selver, Carlsbad, CA (US); Valentin Siderskly, Briarcliff Manor, NY (US); Fernando Jose Isaza, Carlsbad, CA (US)

(73) Assignee: KONINKLKKE PHILIPS N.V.

(21) Appl. No.: 14/388,521

(22) PCT Filed: Mar. 30, 2013
(86) PCT No.: PCT/IB2013/052569

§ 371 Date: Sep. 26, 2014

Related U.S. Application Data
(60) Provisional application No. 61/618,043, filed on Mar. 30, 2012.

(51) Int. Cl. A61M 16/00; A61M 16/20

(52) U.S. Cl. CPC A61M 16/0069; A61M 16/0003; A61M 16/20; A61M 2016/0015; A61M 2205/3327; A61M 2205/505; A61M 2205/702; A61M 2230/40; A61M 2016/0027
USPC 128/204.23

(57) ABSTRACT

A system, method and non-transitory computer readable storage medium for retrieving a breathing reference value, assessing a breathing value of a test subject via a ventilator, identifying a difference between the breathing reference value and the breathing value of the test subject and generating a setting adjustment value to adjust a setting on the ventilator based on the identified difference.

In the field of health care, mechanical ventilators may be any machine designed to mechanically move breatheable air into and out of the lungs, thereby providing the mechanism of breathing for a patient who is physically unable to breathe, or breathing insufficiently. Ventilators are primarily used in intensive care medicine, home care, and emergency medicine (e.g., standalone units) and in anesthesia (e.g., a component of an anesthesia machine).

On any given day, 35,000 patients may be on ventilators in the US, and 100,000 patients worldwide. Almost all of these patients would die if ventilators did not exist. Of these ventilated patients, roughly 7-10% will experience complications from the ventilator systems due to errors in system settings and inaccuracies in the assessment of a patient's lung functioning. Accordingly, it is generally a daunting task for the medical personnel to select the proper values of the various ventilator settings in order to provide effective artificial ventilation for a specific patient at a specific time period. The settings on the ventilator may relate to values in tidal volume, respiratory rate, pressure readings, etc.

An exemplary embodiment is directed to a method for retrieving a breathing reference value, assessing a breathing value of a test subject via a ventilator, identifying a difference between the breathing reference value and the breathing value of the test subject and generating a setting adjustment value to adjust a setting on the ventilator based on the identified difference.

A further exemplary embodiment is directed to a system having a data retrieval component for retrieving a breathing reference value and a processing component configured to assess a breathing value of a test subject via a ventilator, identify a difference between the breathing reference value and the breathing value of the test subject and generate a setting adjustment value to adjust a setting on the ventilator based on the identified difference.

A further exemplary embodiment is directed to a non-transitory computer readable storage medium including a set of instructions that are executable by a processor. The set of instructions are operable at least to retrieve a breathing reference value, assess a breathing value of a test subject via a ventilator, identify a difference between the breathing reference value and the breathing value of the test subject and generate a setting adjustment value for adjusting a setting on the ventilator based on the identified difference.

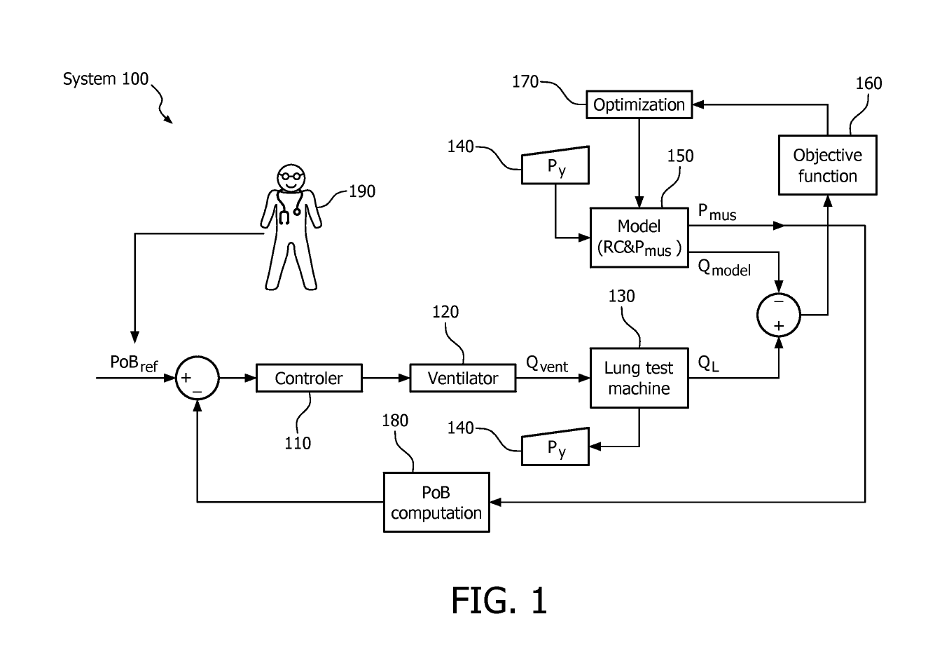

FIG. 1 shows an exemplary closed-loop system for assessing the breathing effort of a ventilated patient and providing appropriate setting values according to an exemplary embodiment described herein.

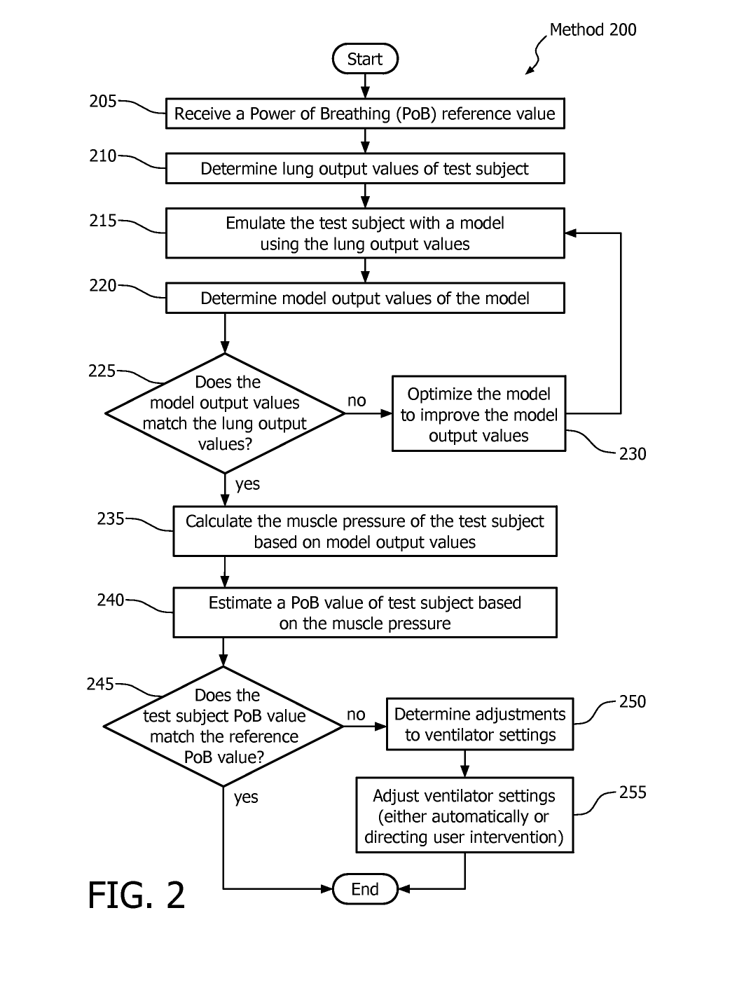

FIG. 2 shows an exemplary method for assessing the breathing effort of a ventilated patient and providing appropriate setting values according to an exemplary embodiment described herein.

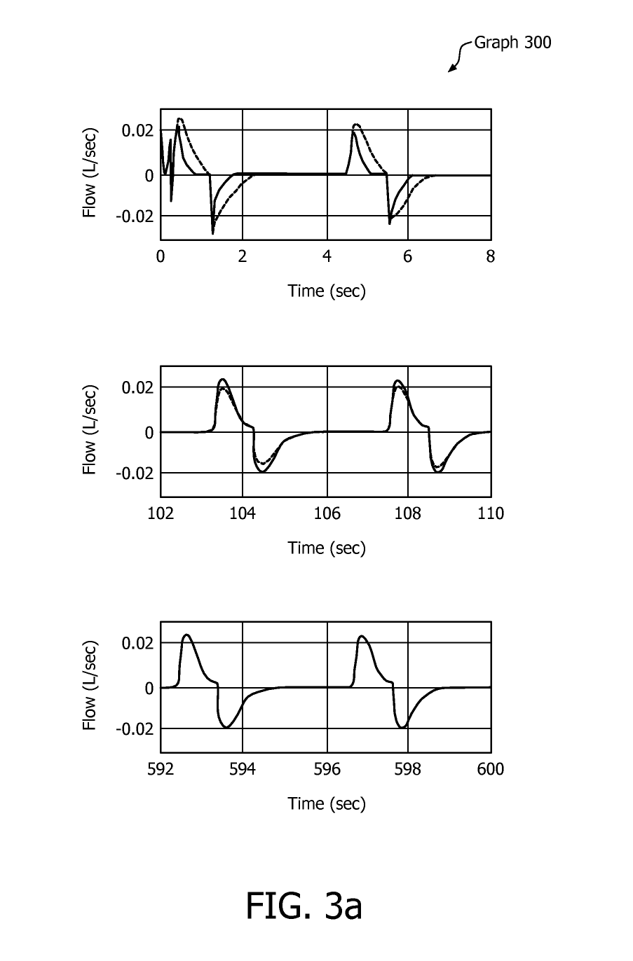

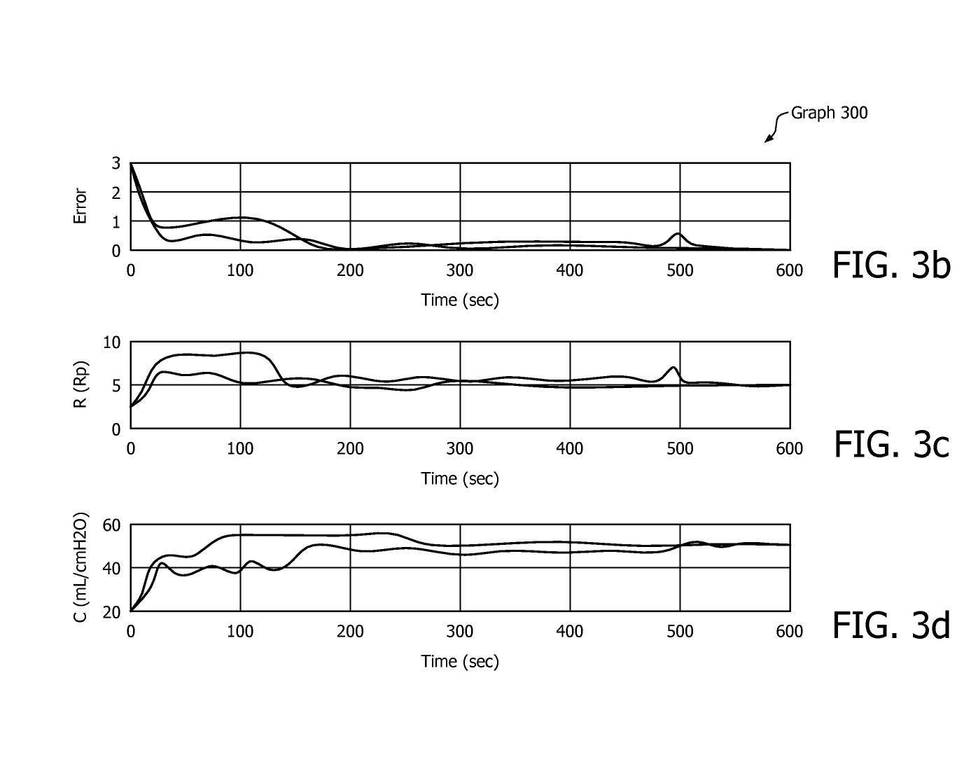

FIGS. 3a-3d show exemplary graphs for the real-time estimations of airway resistance (R) of a tested lung and compliance (C) of the lung according to an exemplary embodiment described herein.

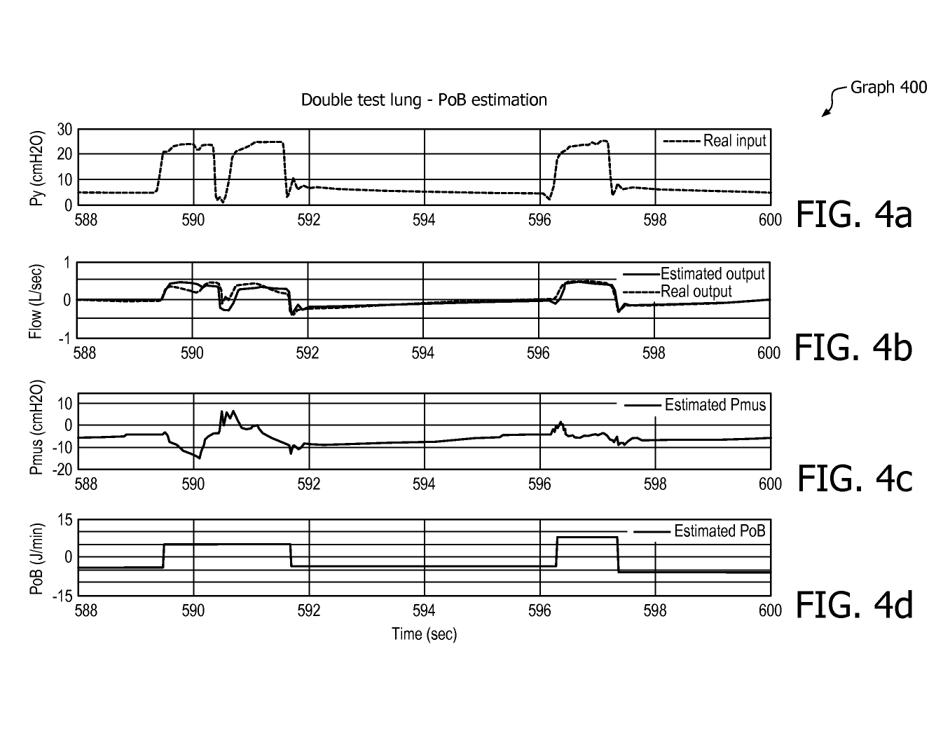

FIGS. 4a-4d show exemplary graphs for the real-time estimations of thoracic muscle pressure (Pₘᵤₛ) and power of breathing value (PoB) during a lung test according to an exemplary embodiment described herein.

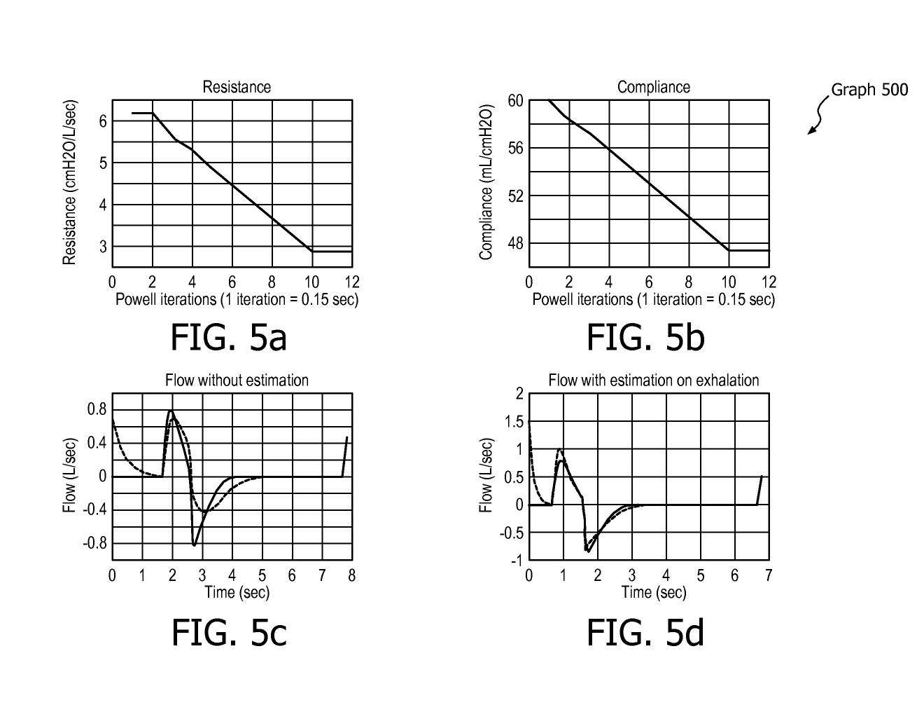

FIGS. 5a-5d show exemplary graphs for a fast (e.g., under 2 seconds) real-time estimation R and C values for a tested lung according to an exemplary embodiment described herein.

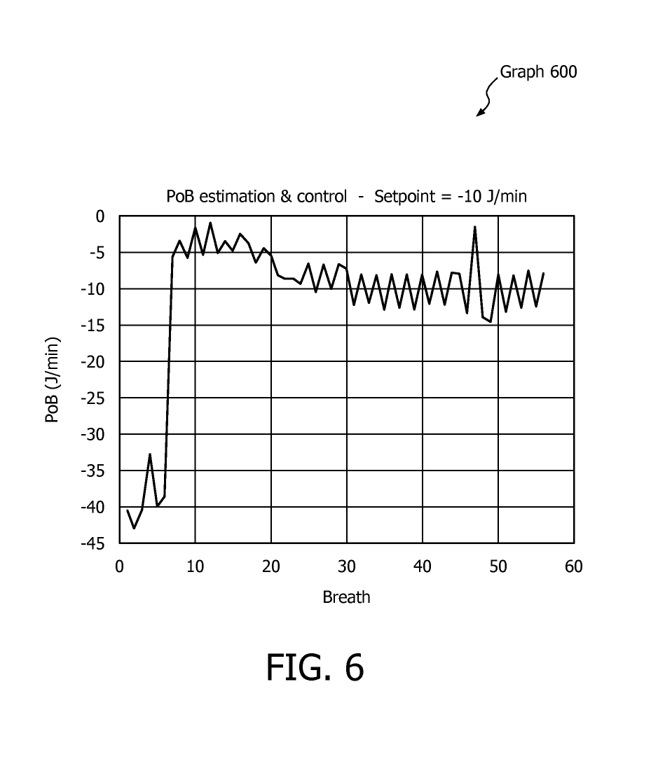

FIG. 6 shows an exemplary graph of the real-time performance by a PoB controller according to an exemplary embodiment described herein.

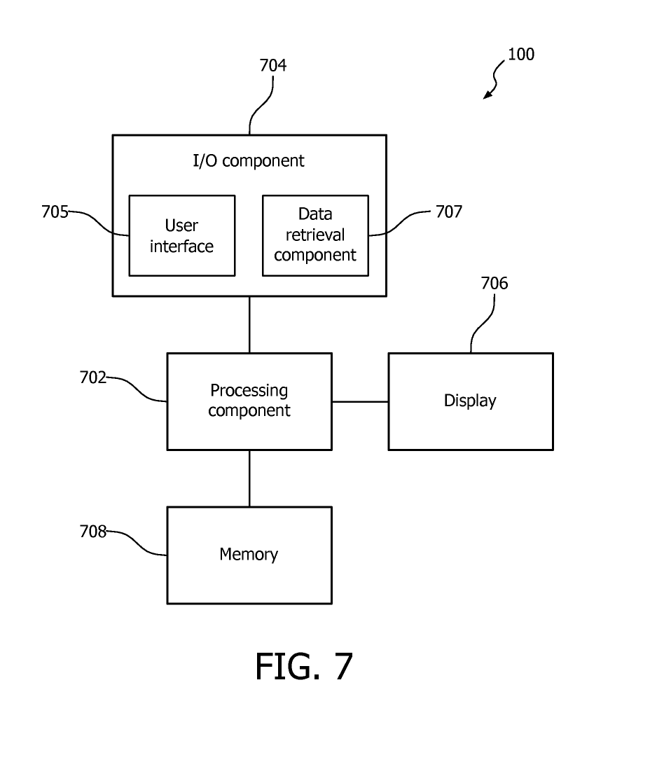

FIG. 7 shows a schematic diagram of the system according to an exemplary embodiment

The exemplary embodiments may be further understood with reference to the following description of exemplary embodiments and the related appended drawings, wherein like elements are provided with the same reference numerals. The exemplary embodiments are related to systems and methods for assessing a ventilated patient's Power of Breathing (“PoB”). A patient's PoB may depend on any number of variables, such as, but not limited to, the quality of the lungs, the strength of the lungs, etc. Furthermore, the exemplary systems and methods provide supportive information for the ventilator system such as system settings and values.

Specifically, the exemplary systems and methods utilize a closed-loop feedback control system in order to automatically and non-invasively assess how much effort the ventilated patient is making. The assessment of this effort gives the user (e.g., clinician, care provider, hospital personnel, etc.) with the appropriate setting values for the ventilator system to make the decision on selecting functions of the ventilator as well as any adjustments to these functions. Alternatively, the exemplary systems and methods described herein may also automatically perform the selection and adjustment of these functions without user intervention.

As will be described in greater detail below, these exemplary systems and methods use optimization algorithms in conjunction with the closed-loop control system to determine lung variables of the patient, such as pressure and volume. Based on these determined variables, the systems and methods will provide adjustments to ventilator settings to achieve a desired breathing level. Furthermore, the assessments performed by the systems and methods allow for a user to easily identify candidates for ventilation weaning (e.g., the reduction of a patient's dependency on the ventilator system).

FIG. 1 shows an exemplary closed-loop system 100 for assessing the breathing effort of a ventilated patient and providing appropriate setting values according to an exemplary embodiment described herein. The architecture of the system 100 includes a controller 110, a ventilator 120, a patient 130, a circuit model of the lung 150, and an optimizer 170. It should be noted that while FIG. 1 depicts a “lung test machine” at 130, this component may be either attached to the patient during medical practice or unattached to a patient for calibrating the system 100. In other words, a lung test machine 130 may act as the lungs of the patient during performance testing and calibration of the system 100. For the sake of simplicity, the lung test machine of FIG. 1 may be referred to as the patient 130. Accordingly, the system 100 allows for non-invasive assessment of the lung strength and lung quality of the patient 130 while offering corresponding support information for adjusting a ventilator 120.

The exemplary controller 110 may be, for example, a proportional integral controller. However, the controller 110 may also be any controller with good stability margins for tracking and disturbance rejection. It should be noted that while the controller 110 and ventilator 120 are illustrated as separate components within the system 100, these components may be integrated into a single component.

The exemplary feedback control system 100 of FIG. 1 depicts a physician 190 setting a reference value for the desired power of breathing (POB_{ref}). Specifically, the PoB_{ref} value set by the physician 190 may be entered into the controller 110. The controller 110 adjusts the settings of the ventilator 120, thereby adjusting the airflow from the ventilator value (Qᵥₑₙₜ) When the Qᵥₑₙₜ value reaches the patient 130, the patient 130 responds by providing an airflow in the lung (Q_{L}) value and a pressure at wye (P_{Y}) value 140. The P_{Y} value 140 is then supplied to the circuit model of the lung 150, wherein the model 150, in turn, provides an airflow as computed by the model 150 (Q_{model}).

The circuit model 150 is a simple mathematical model, such as a hydraulic RC circuit, to emulate the lungs of the patient 130 in real-time based on airway resistance of lungs (R) and compliance of lungs (C). Specifically, the model 150 accurately emulates the patient's lungs whenever the R and C values of the model 150 correspond to those of the patient 130.

In order to obtain the patient's R and C values in real-time, the exemplary system 100 utilizes optimization algorithms of the optimizer 170. For instance, if the Q_{model} value and the Q_{L} value are not equal (e.g., the model 150 does not emulate the patient 130), then the error difference may be supplied to an optimizing algorithm of the optimizer 170. Accordingly, the optimizer 170 may use this error difference as a point of an objective function 160 to be minimized. The construction of the objective function 160 in time by the optimizer 170 may be referred to as “gradient-free optimization.” It should be noted that this particular technique for optimization is merely an example of one algorithm used by the optimizer 170. Any number of parameter estimation algorithms can also be implemented in order to provide adequate results in real-time.

Regardless of the specific algorithms implemented by the optimizer 170, the output of the optimizer 170 is a set of new values for R and C. These new R and C values are then supplied to the model 150 and the model 150 is thus updated accordingly. Using the R and C values, the model 150 estimates the thoracic muscle pressure (Pₘᵤₛ). For instance, the model 150 can solve for Pₘᵤₛ based on the following equation:

Pₘᵤₛ=Q_{L}·R+V_{L}/C−P_{Y}.

Once the Pmus is estimated, the PoB is computed at 180 and provided back to the controller 110. For instance, the PoB may be calculated using the following equation:

PoB=integrate(Pₘᵤₛ·Q_{L}dt).

At the controller 110, the value of the PoB 180 is then compared to the reference value (PoB_{ref}) set by the physician 190. Accordingly, the error determined from this comparison provides the setting information for appropriate adjustments to the ventilator 120. The adjustments made to the ventilator 120 may be performed automatically by the controller 110 (e.g., without user intervention), or alternatively, the controller 110 may provide the user with adjustment instructions for manually selecting values on the ventilator 120.

As described above, the exemplary system 100 allows for the user (e.g., physician 190) to work on a higher strategic level and eliminate the need to be troubled with the “pipes and knobs” of the ventilator 120. One example of a strategic decision made by the physician 190 could be that the patient 130 needs to be breathing no harder than 10 J/min (e.g., Joules of thoracic muscle work per minute). Using this high level setting form the physician 190, the exemplary system 100 accomplishes the task of automatically guiding the patient to breathe at 10 J/min without any need for the physician to adjust or control the ventilator 120. As noted above, another embodiment of the system 100 allows for the physician to be “in the loop” as the controller 110 provides the physician 190 with appropriate ventilator setting instructions. Accordingly, the physician 190 may then ultimate decide whether to accept or reject the setting decisions (e.g., knob settings) provided by the controller 110.

FIG. 2 shows an exemplary method 200 for assessing the breathing effort of a ventilated patient 130 and providing appropriate setting values according to an exemplary embodiment described herein. It should be noted that method 200 will be discussed with reference to system 100 and related components of the system 100 illustrated in FIG. 1.

As detailed above, the system 100 allows for users (e.g., clinicians, hospital personnel, etc.) to assess the PoB of the patient 130 and suggest adjustments to the operation of the ventilator 120.

According to one of the exemplary embodiments, the method 200 may be performed by an add-on embedded component to existing services, such as an anesthesia machine or monitor (e.g., the Philips ALPS platform or Philips NM3 platform). Alternatively, the method 200 may be performed by a stand-alone ventilator within a hospital (e.g., in an intensive care unit (“ICU”), emergency room (“ER”), operating room (“OR”), etc.).

In step 205, the system 100 receives a PoB reference value from the user (e.g., physician 190). While the exemplary system 100 describes a physician 190 as the source of the PoB reference value, this information may be retrieved from any source, either manually (e.g., via other personnel) or automatically (e.g., via a Clinical Decision Support (“CDS”) system).

In step 210, the system 100 determines the lung output values of a test subject. These output values include a pressure value P_{Y} 140 and an airflow value Q_{L} from the test subject. As noted above, the test subject may either be the patient 190 under medical care or lung test machine used to calibrate the system 100.

In step 215, the system 100 emulates the lungs of the test subject with the model 150. Specifically, the model 150 receives the pressure value P_{Y} 140 of the test subject as an input and emulates the lungs based on this value. As described above, the model 150 may be a mathematical hydraulic RC circuit used to emulate the lungs in real-time.

In step 220, the system 100 determines the model output values from the model 150 as it emulates the lungs. These output values include an airflow value Q_{model} from the model 150.

In step 225, the system 100 compares the airflow value Q_{L} of the test subject to the airflow value Q_{model} of the model 150. If the values match, the method 200 may advance to step 235. However, if the values do not match, the method 200 advances to step 230 for optimization.

In step 230, the system 100 the optimizer 170 receives the difference between the airflow value Q_{L} of the test subject to the airflow value Q_{model} of the model 150, and uses this difference as a point of an objective function 160 to be minimized. Using optimization algorithms, the optimizer 170 sets new values for airway resistance R of the model 150 and lung compliance C of the model 150. These new values are used to update the model 150, and the method 200 returns to step 215 to emulate the test subject.

In step 235, the system 100 calculates the thoracic muscle pressure (Pₘᵤₛ) of the test subject based on the matching model output values. As detailed above, the system 100 may utilize the Pₘᵤₛ model equation to solve for Pₘᵤₛ using the R and C values. It should be noted that any of the variables in these equations would change over time.

In step 240, the system 100 estimates the PoB of the test subject based on the calculated Pₘᵤₛ of step 235. As detailed above, the system 100 may utilize the PoB equation 180 to solve for the PoB of the test subject using the Pₘᵤₛ and Q_{L} values.

In step 245, the system 100 compares the PoB of the test subject to the reference PoB. If the PoB values match, then the system 100 has achieved the breathing pressure and functions desired by the physician 190. However, if the PoB values do not match, the method 200 advances to step 250 for optimization.

In step 250, the system 100 determines adjustments to the settings of the ventilator 120. These adjustments may include changing settings such as tidal volume, respiratory rate, pressure readings, airflow, etc. Furthermore, any adjustments to the settings may include changes to an operating mode of the ventilator 120. One skilled in the art would understand that these various modes may come in any number of delivery concepts, such as, but not limited to, volume controlled continuous mandatory ventilation, volume controlled intermittent mandatory ventilation, pressure controlled continuous mandatory ventilation, pressure controlled intermittent mandatory ventilation, continuous spontaneous ventilation, high frequency ventilation systems, etc.

In step 255, the system 100 adjusts the settings of the ventilator 120 according to the determined adjustments of step 250. As detailed above, the adjustment performed on the operation of the ventilator 120 may be either automatically performed by the system 100, or alternatively, performed by the user as directed by the system 100. Once the settings of the ventilator 120 have been adjusted (automatically or manually), the system 100 has achieved the breathing pressure and functions desired by the physician 190.

The exemplary method 200 described above is merely an example of any number of steps performable by the system 100 and related components of the system 100. Accordingly, the system 100 is not limited to steps recited in exemplary method 200, and may perform additional steps or less steps than steps 210-255 and any sub-steps, and in any order.

FIGS. 3a-3d show exemplary graphs 300 for the real-time estimations of airway resistance (R) of a tested lung and compliance (C) of the lung according to an exemplary embodiment described herein. FIG. 3a demonstrates how, over time, the airflow as computed by the model 150 (Q_{model}) comes to approximate the airflow in the patient's lung (Q_{L}) very well. FIG. 3b illustrates the error difference between the two signals of the upper portion. Furthermore, FIGS. 3c and 3d represent the R and C values, respectively. Both the R and C values may converge to the correct values as a priori has set them via a lung test machine. Accordingly, the optimization algorithms do not require these two set values.

FIGS. 4a-4d show exemplary graphs 400 for the real-time estimations of thoracic muscle pressure (Pmus) and power of breathing value (PoB) during a lung test according to an exemplary embodiment described herein. FIG. 4a illustrates pressure at wye as a function of time, P_{Y}(t). FIG. 4b illustrates the estimated airflow output (solid line) and the real output (dashed line), where the approximation in real-time is acceptable. FIGS. 4c and 4d illustrate the real-time non-invasive estimation of Pₘᵤₛ and PoB, respectively.

FIGS. 5a-5d show exemplary graphs 500 for a fast (e.g., under 2 seconds) real-time estimation R and C values for a tested lung according to an exemplary embodiment described herein. While FIG. 3 depicted the real-time estimation over a longer time period (e.g., 500 seconds), FIGS. 5a-5d accomplishes the same task in under 2 seconds. The values of R and C quickly converge to the correct values, as detailed in FIGS. 5a and 5b, respectively. FIGS. 5c and 5d illustrate the convergence of the Q_{model} and Q_{L} both without parameter estimation and with parameter estimation, respectively.

FIG. 6 shows an exemplary graph 600 of the real-time performance by the PoB controller 110 according to an exemplary embodiment described herein. As described above, the PoB_{ref} may be set by the physician 190. According to FIG. 6, the PoB_{ref} is set to −10 J/min, wherein pulling or pushing of air provides the change in sign on this value. Within 25 breaths (by either the patient 130 or a lung machine), the desired PoB_{ref} is achieved.

FIG. 7 shows a schematic diagram of the system 100 according to an exemplary embodiment including a processing component (e.g., processor 702), an input/output component 704, a display 706 and a non-transitory computer readable storage medium (e.g., memory 708). The processor 702 that is capable of processing data entered via the input/output component 704, such as data received from a user interface 705 and a data retrieval component 707. The data may include a breathing reference value for identifying any error difference between the breathing value of an exemplary circuit model and the breathing value of a test subject. The display 706 may be used to display model information, various measurements and reading from the patient, machine setting values, setting adjustment valve, operating instructions to the user, etc. For instance, the displayed modeling information may be loaded from the memory 708, which includes a database storing the computerized representations of industry-accepted circuit models, guidelines, protocols and/or workflows. The memory 708 also stores information that has been updated with patient-specific information. The user interface 704 may include a mouse to point and click on items on the display 706, a touch display and/or a keyboard. The memory 708 may be any known type of computer-readable storage medium. It will be understood by those of skill in the art that the system 100 is, for example, a personal computer, a server, or any other processing arrangement.

Those skilled in the art will understand that the above-described exemplary embodiments may be implemented in any number of manners, including, as a separate software module, as a combination of hardware and software, etc. For example, the system 100 and related components may be a program containing lines of code stored on a non-transitory computer readable storage medium that, when compiled, may be executed on a processor. It should also be apparent from the above description that the exemplary embodiments allow the processing device to operate more efficiently when a user implements the system 100, e.g., by improving patient breathing assessment for health care professionals, by automatically suggesting one or more ventilator settings based on the assessed effort, by contributing in the identification of candidates for ventilation weaning, by assisting the health care professionals with the weaning process, etc.

It is noted that the claims may include reference signs/numerals in accordance with PCT Rule 6.2(b). However, the present claims should not be considered to be limited to the exemplary embodiments corresponding to the reference signs/numerals.

It will be apparent to those skilled in the art that various modifications may be made in the present invention, without departing from the spirit or the scope of the invention. Thus, it is intended that the present invention cover modifications and variations of this invention provided they come within the scope of the appended claims and their equivalents.

1. (canceled)

2. (canceled)

3. (canceled)

4. (canceled)

5. (canceled)

6. (canceled)

7. (canceled)

8. A system, comprising:
a data retrieval component for retrieving a Power of Breathing reference value; and
a processing component configured to assess a Power of Breathing value of a test subject via a ventilator, identify a difference between the Power of Breathing reference value and the Power of Breathing value of the test subject and generate a setting adjustment value to adjust a setting on the ventilator based on the identified difference.

9. The system of claim 8, wherein the processing component is further configured to automatically adjust the setting on the ventilator in accordance with the setting adjustment value.

10. The system of claim 8, wherein the processing component is configured to assess the Power of Breathing value of the test subject by:
determining a lung output pressure of the test subject;
emulating the test subject with a model using the lung output pressure; and
measuring a breathing value of the model.

11. The system of claim 10, wherein the processing component is further configured to identify an error difference between the breathing value of the model and a breathing value of the test subject and optimizes the model based on the identified error difference.

12. The system of claim 11, wherein the processing component is configured to optimizes the model by:
minimizing the identified error difference using an objective function;
calculating at least one new variable within the model; and
updating the model with the at least one new variable.

13. The system of claim 8, wherein the system is a closed-loop feedback controlled system.

14. The system of claim 8, wherein the processing component is further configured to identify the test subject as a candidate for reduced dependency on the ventilator based on the assessed Power of Breathing value of the test subject.

15. A non-transitory computer readable storage medium including a set of instructions that are executable by a processor controlling the system of claim 8, the set of instructions being operable at least to:
retrieve a Power of Breathing reference value;
assess a Power of Breathing value of a test subject via a ventilator;
identify a difference between the Power of Breathing reference value and the Power of Breathing value of the test subject; and
generate a setting adjustment value for adjusting a setting on the ventilator based on the identified difference.

16. The non-transitory computer readable storage medium of claim 15, wherein the set of instructions are further operable to:
automatically adjust the setting on the ventilator in accordance with the setting adjustment value.

17. The non-transitory computer readable storage medium of claim 15, wherein assessing the Power of Breathing value of the test subject includes:
determining a lung output pressure of the test subject;
emulating the test subject with a model using the lung output pressure; and
measuring a breathing value of the model.

18. The non-transitory computer readable storage medium of claim 17, wherein the set of instructions are further operable to:
identify an error difference between the breathing value of the model and a breathing value of the test subject; and
optimize the model based on the identified error difference.

19. The non-transitory computer readable storage medium of claim 18, wherein optimizing the model includes:
minimizing the identified error difference using an objective function;
calculating at least one new variable within the model; and
updating the model with the at least one new variable.

20. The non-transitory computer readable storage medium of claim 15, wherein the set of instructions are further operable to:
identify the test subject as a candidate for reduced dependency on the ventilator based on the assessed Power of Breathing value of the test subject.
